(19) United States
(12) Patent Application Publication
(10) Pub. No.: US 2003/0161868 A1
(43) Pub. Date: Aug. 28, 2003

(54) Dermal therapeutic system containing 2-(3-benzophenyl)-propionic acid or '0-(2,6-dichloranilino)-phenyl!-ethanoic acid

(76) Inventors: Gunter Cordes, Langenfeld (DE); Ulrike Vollmer, Langenfeld (DE)

Correspondence Address: Ronald R Santucci, Frommer Lawrence & Haug, 745 Fifth Avenue, New York, NY, 10151, US

(21) Appl. No.: 10/332,221

(22) PCT Filed: Jul. 5, 2001
(86) PCT No.: PCT/EP01/07712

(30) Foreign Application Priority Data
Jul. 5, 2000 (DE) 100 32 537.8

(51) Int. Cl. A61K 9/70; A61K 31/192

(52) U.S. Cl. 424/449; 514/570

(57) ABSTRACT

The invention relates to a dermal therapeutic system having a cover layer, an adhesive matrix having a content of, as active ingredient, 2-(3-benzophenyl)propionic acid or [o-(2,6-dichloroanilino)phenyl]acetic acid or a derivative thereof customary in pharmacy, and a removable protective layer, characterised in that the adhesive matrix is an acrylate copolymer adhesive matrix.

2-(3-Benzophenyl)propionic acid, as a substance, was patented as early as 1968. Since then, it has been found to be highly advantageous in the therapy of acute forms of arthritis, including gout attack, chronic forms of arthritis, especially rheumatoid arthritis (chronic polyarthritis), and also ankylosing spondylitis (Bechterew's disease) and other inflammatory rheumatic conditions of the spine, irritation conditions in the case of degenerative joint and spine diseases (arthroses and spondylarthroses), soft tissue rheumatism, painful swellings or inflammations after injuries or operations and also other non-rheumatic pain conditions and dysmenorrhoea. Because gastric and intestinal ulcers, gastrointestinal complaints such as nausea, vomiting, heartburn, stomach pain, sensation of bloatedness, constipation or diarrhoea frequently occur as side-effects when this class of substance and also, therefore, 2-(3-benzophenyl)propionic acid are administered perorally and because a large proportion of the indications are also amenable to treatment topically, especially in the case of irritation conditions in degenerative joint and spine diseases (arthroses and spondylarthroses), soft tissue rheumatism, painful swellings or inflammations after injuries and operations, the substance is also employed in topical formulations such as creams, ointments, gels, sprays etc. For that purpose, the 2-(3-benzophenyl)propionic acid released from the formulation penetrates the skin barrier and, by virtue of the acidic environment caused by the inflammation, accumulates in the inflamed soft tissue, where it produces a topical effect in the painful and inflamed region of that part of the body.

The penetration of pharmaceutical substances through the skin is largely governed by the physicochemical properties of the substance—the octanol/water partition coefficient and molecule size basically playing a role therein (Potts R O, Guy R H in: Gurny R, Teubner A; Dermal and transdermal drug delivery, Wiss. Verlagsges. Stuttgart (1993)). Because those parameters cannot be influenced without making modifications to the molecule, there are basically only two possibilities for increasing the penetration rate:

1. facilitating diffusion by means of the addition of penetration accelerators or employing electric potential (iontophoresis)

2. increasing the concentration of pharmaceutical substance in the base, even to beyond the limit of solubility (supersaturation).

As penetration accelerators there are used, inter alia, fatty acids, fatty alcohols, simple and polyhydric alcohols, laurocapram and surfactants. However, many of those substances act by disrupting the barrier function of the skin and consequently have to be categorised as being more or less irritating to the skin. Nevertheless, many such systems are described in patent specifications (cf. DE 19830649, WO 96229988 etc.).

Tolerability is better when systems are used in which the active ingredient is present in supersaturated form. Usually the maximum flux of a substance through the skin is limited by its solubility in the horny layer (stratum corneum), which constitutes the main penetration barrier. The said saturation concentration will be achieved when the active ingredient is present in the vehicle, for example in the matrix of the transdermal system, in a concentration that also corresponds to its solubility in that vehicle. A possibility for further increasing that so-called maximum thermodynamic activity consists in incorporating the pharmaceutical substance in a concentration exceeding its solubility in the vehicle. That is possible, for example, by incorporating 2-(3-benzophenyl)propionic acid in acrylate copolymers (DE 19843027). However, it is necessary for supersaturation to be sensitively adjusted so that the supersaturations are as high as possible but are also as stable as is necessary, because supersaturated systems are known to be metastable and recrystallisation during storage causes them to change to the saturated state. It is then disadvantageous that, because of the crystallisation, such systems lead to product complaints, resulting from shortcomings in appearance and also from a lack of adhesive strength. It is likewise necessary for there to be close contact between a dermal system and the skin in order to obtain an effective amount of 2-(3-benzophenyl)propionic acid in the target region of the inflamed soft tissue.

It has now been found that incorporating 2-(3-benzophenyl)propionic acid in a very specific acrylate copolymer achieves supersaturation that is so stable that an effective product is obtained without having to add penetration accelerators, as well as optimum adhesion to the skin such that, even with close contact between the dermal system and the outer barrier of the skin for several days up to a maximum of one week, the system can be removed at any time, without resulting in painful sensations or skin irritations. Consequently, the adhesive strength of the dermal system according to the invention makes possible a significantly longer period of wear than, for example, products that are on the market which comprise water-containing preparations of the cataplasm or poultice kind, as well as substantially longer contact than conventional topical formulations such as creams, gels or a spray, which can be removed as a result of contact with water or with clothing.

A number of solvent-based acrylate copolymers, as are made available, for example, by the company National Starch & Chemical, BV, Zutphen, Netherlands under the trade-name Durotak, have been tested with regard to their adhesion properties. The table that follows indicates the copolymer composition:

| Monomer | | | | | |
|---|---|---|---|---|---|
| composition | Durotak | Durotak | Durotak | Durotak | Durotak |
| of adhesive | 387-2825 | 387-2054 | 87-2852 | 387-2516 | 87-2070 |
| Butyl acrylate | | X | | | |
| Methyl | | | | | |
| methacrylate | | | | | |
| 2-Ethylhexyl | X | X | X | X | X |
| acrylate | | | | | |
| Methyl acrylate | | | X | | |
| Vinyl acetate X | X | | X | X | |
| Acrylic acid | X | X | X | | X |
| Glycidyl | | | | X | X |
| methacrylate | | | | | |
| 2-Hydroxymethyl | | | | X | |
| acrylate | | | | | |
| Adhesion | remnants | remnants | good | adheres too | adheres too |
| properties | on skin | on skin | | weakly | weakly |
| | when | when | | | |
| | removed | removed | | | |

As can be seen, the properties for wear are achieved only by using an adhesive based on 2-ethylhexyl acrylate, methyl methacrylate and acrylic acid, for example by Durotak 2852, which is surprising. The company National Starch & Chemical makes available slight modifications of that adhesive (Durotak 387-2287, 387-2353), although the above-mentioned adhesive Durotak 87-2852 leads to the best result, with specific, unknown interactions taking place between the active ingredient and acrylate copolymer.

In addition, the support or cover layer of the matrix plays an important part in the properties for wear. Because the dermal system has to be applied to joints, a high degree of flexibility is necessary. Various materials, encompassing non-wovens, foams, films and wovens, have undergone testing. It was important, inter alia for tolerability, that the support used should have good permeability to water vapour. A longitudinally and transversally resilient woven polyester which is available in white or skin colours (company Karl Otto Braun, Germany) was found to be optimal.

As protective film there can be used a siliconised polyester film known to the person skilled in the art, for example Hostaphan RN 100 from Diafoil, Hoechst, Germany, easy/easy, which must not be too thin (a layer at least 36 μm thick, preferably 100 μm thick) so that a system of a large size such as from 70 to 140 cm², preferably of 90 cm², can still be handled well by the patient.

The dermal therapeutic systems according to the invention are preferably so arranged that they consist of a cover layer impermeable to the active ingredient, an active-ingredient-containing adhesive layer adhering to the cover layer, and a removable protective layer.

This most simple form of a TDS can be produced in a manner well known to the person skilled in the art, by mixing a solution of the adhesive in a low-boiling solvent with the active ingredient, applying the mixture evenly to a removable protective layer, removing the solvent by heating, and covering the resulting product with a support. The active-ingredient-containing adhesive layer applied has a thickness of from 20 to 500 μm.

The following exemplary embodiments serve to illustrate the invention in greater detail:

### EXAMPLE 1

To 30.83 g of a 36% (w/w) solution of an acrylate adhesive (Durotak 87-2852, National Starch & Chemical B. V., NL-Zutphen) there is added a solution of 2.78 g of 2-(3-benzophenyl)propionic acid in 5.6 g of 2-propanol. The solution is homogenised by stirring for one hour and is then spread out, using a doctor blade, onto a siliconised, 100 μm-thick polyester film (FL 2000 100μ 1-S, Rexam Release B. V., NL-Apeldoorn) in a wet-layer thickness of 260 μm. After drying (1 h at 40° C. and 50 min at 80° C.), the clear and homogenous laminate is lined with a woven polyester (M02/97, white, K. O. Braun, D-Wolfstein) without stretching. A patch 90 cm2 in size, at a matrix weight of 55.6 g/m², contains 100 mg of 2-(3-benzophenyl)propionic acid.

### EXAMPLE 2

To 17.46 g of a 35% (w/w) solution of an acrylate adhesive (Durotak 87-2852, National Starch & Chemical B. V., NL-Zutphen) there is added a solution of 2.08 g of 2-(3-benzophenyl)propionic acid and 0.21 g of neohesperidine DC in 4.17 g of isopropanol. The solution is homogenised by stirring for one hour and is then spread out, using a doctor blade, onto a siliconised, 100 μm-thick polyester film (FL 2000 100μ 1-S, Rexam Release B. V., NL-Apeldoorn) in a wet-layer thickness of 270 μm. After drying (1 h at 40° C.), the slightly cloudy laminate is lined with a longitudinally and transversely resilient woven polyester (K. O. Braun, D-Wolfstein). A patch 90 cm² in size, at a matrix weight of 55.6 g/m², contains 125 mg of 2-(3-benzophenyl)propionic acid.

As Table 2 shows, the Example comprising neohesperidine DC does not have modified adhesive properties compared to the corresponding formulations without neohesperidine DC.

TABLE 2
Effect of neohesperidine DC (Example 4, n = 3) on adhesive properties of
transdermal therapeutic systems

| | 2-(3-Benzophenyl) | 2-(3-Benzophenyl)propionic |
|---|---|---|
| | propionic acid TDS | acid TDS with addition of |
| | without addition of | 2.5 % neohesperidine DC |
| | neohesperidine DC | (based on the matrix) |
| Adhesive strength | 6.8 ± 0.6 | 6.2 ± 0.4 |
| [N/25 mm] | | |
| Separating force | 0.137 ± 0.012 | 0.127 ± 0.025 |
| [N/25 mm] | | |

1. Dermal therapeutic system having a cover layer, an adhesive matrix having a content of, as active ingredient, 2-(3-benzophenyl)propionic acid or [o-(2,6-dichloroanilino)phenyl]-acetic acid or a derivative thereof customary in pharmacy, and a removable protective layer, characterised by an adhesive matrix
(a) which is obtainable by free-radical-copolymerisation of 2-ethylhexyl acrylate, methyl acrylate and acrylic acid as sole monomers, or
(b) in which the acrylate copolymer consists of units originating exclusively from 2-ethylhexyl acrylate, methyl acrylate and acrylic acid as monomers, the adhesive matrix being free from penetration accelerators.

2. Dermal therapeutic system according to claim 1, characterised by 2-(3-benzophenyl)propionic acid in the form of the pure enantiomer or the racemate.

3. Dermal therapeutic system according to at least one of the preceding claims, characterised by a content of 2-(3-benzophenyl)propionic acid in a concentration of from 0.1 to 30% by weight and especially from 15 to 25% by weight, in each case based on the weight of the adhesive matrix together with active ingredient.

4. Dermal therapeutic system according to claim 1 (a), characterised in that it is obtainable from acrylic acid, methyl acrylate and 2-ethylhexyl acrylate in a ratio of acrylic acid:methyl acrylate of about 1:4 and a ratio of acrylic acid:2-ethylhexyl acrylate of from 1:9 to 1:10 (in each case on a molar basis or weight basis).

5. Dermal therapeutic system according to claim 1 (b), characterised by units originating from acrylic acid and methyl acrylate in a ratio of about 1:4 and by units originating from acrylic acid and 2-ethylhexyl acrylate in a ratio of from 1:9 to 1:10, in each case on a molar basis or weight basis.

6. Dermal therapeutic system according to at least one of the preceding claims, characterised by an adhesive matrix layer thickness of from 20 to 500 μm.

7. Dermal therapeutic system according to at least one of the preceding claims, characterised in that it does not comprise a permeation accelerator.

8. Dermal therapeutic system according to at least one of the preceding claims, characterised by a longitudinally and transversely resilient woven cover layer.

9. Dermal therapeutic system according to claim 8, characterised by a woven polyester cover layer.

10. Dermal therapeutic system according to claim 8 and/or 9, characterised by a woven polyester of warp threads and weft threads of polybutylene terephthalate or polyethylene terephthalate.
